SEQUENCE LISTING (12 sequences)

SEQ ID NO: 1 - protein, 471 aa, Artificial Sequence
  Humanized HC1
MDPKGSLSWRILLFLSLAFELSYGEVQLVESGGGLVQPGGSLKLSCAASGFTFSDAAMYWVRQASGKGLEWVGRIRTKPNNYATYYAASVKGRFTISRDDSKNTAYLQMNSLKTEDTAVYYCTSWSYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 471 aa, Artificial Sequence
  Humanized HC2
MDPKGSLSWRILLFLSLAFELSYGAVQLVESGGGLVQPGGSLKLSCAASGFTFSDAAMYWVRQASGKGLEWVARIRTKPNNYATYYAASVKGRFTISRDDSKNTVYLQMNSLKTEDTAVYYCTSWSYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 471 aa, Artificial Sequence
  Humanized HC3
MDPKGSLSWRILLFLSLAFELSYGAVHLVESGGGLVQPGGSLKLSCAASGFTFSDAAMYWVRQASGKGLEWVARIRTKPNNYATYYAASVKGRFTISRDDSKNTVYLQMNSLKTEDTAVYYCTSWSYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 471 aa, Artificial Sequence
  Humanized HC4
MDPKGSLSWRILLFLSLAFELSYGEVQLVESGGGLVQPGGSLKLSCAASGFTFSDAAIYWVRQASGKGLEWVARIRTKPNNYATYYAASVKGRFTISRDDSNSTAYLQMNSLKTEDTAVYYCTSWSYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 471 aa, Artificial Sequence
  Humanized HC5
MDPKGSLSWRILLFLSLAFELSYGAVQLVESGGGLVQPGGSLKLSCAASGFTFSDAAMYWVRQASGKGLEWVARIRTKPNNYATYYAASVKGRFTISRDDSNSTAYLQMNSLKTEDTAVYYCTSWSYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 239 aa, Artificial Sequence
  Humanized LC1
METDTLLLWVLLLWVPGSTGDIVMTQSPLSLPVTPGEPASISCRSSKSLLHSSGKTYLNWYLQKPGQSPQLLIYWMSNRASGVPDRFSGSGSGTDFTLKISRVEAEDVGVYYCQQFLEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 239 aa, Artificial Sequence
  Humanized LC2
METDTLLLWVLLLWVPGSTGDIVMTQSPLSLPVTPGEPASISCRSSKSLLHSSGKTYLNWYLQKPGQSPQLLIYWMSTRASGVPDRFSGSGSGTDFTLKISRVEAEDVGVYYCQQFLEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 239 aa, Artificial Sequence
  Humanized LC3
METDTLLLWVLLLWVPGSTGDIVMTQSPLSLPVTPGEPASISCRSSKSLLHSSGKTYLNWYLQKPGQSPQLLIYWMSTRASGVPDRFSGSGSGTDFTLKISRVEAEDVGVYYCQQFLEYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 239 aa, Artificial Sequence
  Humanized LC4
METDTLLLWVLLLWVPGSTGDIQMTQSPLSLPVTPGEPASISCRSSKSLLHSSGKTYLNWYLQKPGQSPQLLIYWMSTRASGVPDRFSGSGSGTDFTLKISRVEAEDVGVYYCQQFLEYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 118 aa, Artificial Sequence
  MHC1309HC.4
AVHLVESGGGFVQPTESLKISCAASGFTFSDAAMYWVRQAPGKGLEWVARIRTKPNNYATYYADSVKGRFTISRDDSKSMVYLQMDNLKTEDTAMYYCTSWSYDFDYWGQGVMVTVSS

SEQ ID NO: 11 - protein, 112 aa, Artificial Sequence
  MHC1309LC.1
DIVMTQGALPNPVPSGESASITCQSSKSLLHSSGKTYLNWYLQRPGQSPHLLIYWMSTRASGVSDRLSGSGSGTDFTLKISSVEAEDVGVYYCQQFLEYPLTFGSGTKLEIK

SEQ ID NO: 12 - protein, 166 aa, Homo sapiens
SKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

(19) United States
(12) Patent Application Publication
(10) Pub. No.: US 2023/0287095 A1
(43) Pub. Date: Sep. 14, 2023

(54) HUMANIZED ANTI-EMAP II THERAPEUTIC ANTIBODIES

(71) Applicant: ALLINAIRE THERAPEUTICS, LLC, Cleveland, OH (US)

(72) Inventors: Douglas W.P. Hay, Strafford, PA (US); Suzanne E. Berezovsky, Cleveland Heights, OH (US); David Knight, Tucson, AZ (US); Kexin Huang, Belmont, CA (US); Jordon K. Wang, Belmont, CA (US)

(21) Appl. No.: 18/001,318

(22) PCT Filed: Jun. 28, 2021
(86) PCT No.: PCT/US2021/039389

§ 371 Date: Dec. 9, 2022

Related U.S. Application Data
(60) Provisional application No. 63/045,687, filed on Jun. 29, 2020.

(51) Int. Cl. C07K 16/24

(52) U.S. Cl. CPC C07K 16/24; C07K 2317/24; C07K 2317/92

(57) ABSTRACT

The present disclosure provides monoclonal antibodies that bind to endothelial monocyte activating polypeptide II and methods of treatment using the same.

### CROSS-REFERENCE TO RELATED APPLICATIONS

The present application claims priority under 35 U.S.C. § 119(e) to U.S. Provisional Application No. 63/045,687, filed Jun. 29, 2020, all of which is incorporated herein by reference in its entirety herein.

### BACKGROUND OF THE INVENTION

Endothelial monocyte activating polypeptide II (EMAPII) is a proapoptotic and monocyte chemoattractant cytokine implicated in a wide variety of pathologies, in aggregate having an enormous impact on human health. In particular, EMAPII has been shown to be a mediator of cigarette smoke-induced emphysema. There is a need in the art for novel therapies targeting EMAPII. This disclosure addresses that need.

### BRIEF SUMMARY OF THE INVENTION

In one aspect, the invention provides a humanized anti-endothelial monocyte activating polypeptide II (anti-EMAPII) antibody, comprising: a heavy chain variable domain having an amino acid sequence with 90% or greater sequence identity to any one of SEQ ID NO: 1 to SEQ ID NO: 5; and a light chain variable domain having an amino acid sequence with 90% or greater sequence identity to any one of SEQ ID NO: 6 to SEQ ID NO: 9.

In another aspect, the invention provides a humanized anti-endothelial monocyte activating polypeptide II (anti-EMAPII) antibody, comprising a heavy chain variable domain having an amino acid sequence with 90% or greater sequence identity to any one of SEQ ID NO: 1 to SEQ ID NO: 5.

In yet another aspect, the invention provides a humanized anti-endothelial monocyte activating polypeptide II (anti-EMAPII) antibody, comprising a light chain variable domain having an amino acid sequence with 90% or greater sequence identity to any one of SEQ ID NO: 6 to SEQ ID NO: 9.

In yet another aspect, the invention provides a chimeric anti-endothelial monocyte activating polypeptide II (anti-EMAPII) antibody, comprising a heavy chain variable domain with 90% or greater sequence identity to SEQ ID NO: 10 and a light chain variable domain with 90% or greater sequence identity to SEQ ID NO: 11.

In yet another aspect, the invention provides a recombinant vector, comprising a nucleic acid encoding an amino acid sequence set forth in any one of SEQ ID NO: 1 to SEQ ID NO: 11.

In yet another aspect, the invention provides a cell transformed with the recombinant vector described elsewhere herein.

In yet another aspect, the invention provides a method of producing an antibody, comprising culturing the transformed cell described elsewhere herein under conditions in which the antibody is expressed.

In yet another aspect, the invention provides a pharmaceutical composition comprising the antibody as described elsewhere herein and at least one pharmaceutically acceptable excipient.

In yet another aspect, the invention provides a method of treating chronic obstructive pulmonary disease (COPD) in a subject in need thereof, the method comprising administering to the subject the pharmaceutical composition describe elsewhere herein.

In yet another aspect, the invention provides a method of treating emphysema in a subject in need thereof, the method comprising administering to the subject the pharmaceutical composition described elsewhere herein.

In yet another aspect, the invention provides a method of treating bronchopulmonary dysplasia in a subject in need thereof, the method comprising administering to the subject the pharmaceutical composition described elsewhere herein.

In yet another aspect, the invention provides a method of treating endothelial monocyte activating polypeptide II (EMAPII) mediated disease in a subject in need thereof, the method comprising administering to the subject the pharmaceutical composition as described elsewhere herein.

In certain embodiments, the heavy chain variable domain has an amino acid sequence with 95% or greater sequence identity to any one of SEQ ID NO: 1 to SEQ ID NO: 5; and the light chain variable domain has an amino acid sequence with 95% or greater sequence identity to any one of SEQ ID NO: 6 to SEQ ID NO: 9.

In certain embodiments, the heavy chain variable domain has an amino acid sequence with 99% or greater sequence identity to any one of SEQ ID NO: 2 or SEQ ID NO: 3; and the light chain variable domain has an amino acid sequence with 99% or greater sequence identity to any one of SEQ ID NO: 6 or SEQ ID NO: 9.

In certain embodiments, the heavy chain variable domain has an amino acid sequence with 95% or greater sequence identity to any one of SEQ ID NO: 2 or SEQ ID NO: 3.

In certain embodiments, the light chain variable domain has an amino acid sequence with 95% or greater sequence identity to any one of SEQ ID NO: 6 or SEQ ID NO: 9.

In certain embodiments, the heavy chain variable domain has 95% or greater sequence identity to SEQ ID NO: 10 and the light chain variable domain has 95% or greater sequence identity to SEQ ID NO: 11.

### BRIEF DESCRIPTION OF THE DRAWINGS

The following detailed description of specific embodiments of the invention will be better understood when read in conjunction with the appended drawings. For the purpose of illustrating the invention, the drawings show specific embodiments. It should be understood, however, that the invention is not limited to the precise arrangements and instrumentalities of the embodiments shown in the drawings.

FIG. 1 depicts enzyme linked immunosorbent assays (ELISA) of humanized antibodies with LC1 (SEQ ID NO. 6) against human proEMAPII.

FIG. 2 depicts enzyme linked immunosorbent assays (ELISA) of humanized antibodies with LC2 (SEQ ID NO. 7) against human proEMAPII.

FIG. 3 depicts enzyme linked immunosorbent assays (ELISA) of humanized antibodies with LC3 (SEQ ID NO. 8) against human proEMAPII.

FIG. 4 depicts enzyme linked immunosorbent assays (ELISA) of humanized antibodies with LC4 (SEQ ID NO. 9) against human proEMAPII.

FIG. 5 depicts an ELISA screen of eight antibodies against human proEMAPII.

FIG. 6 depicts an ELISA screen of eight antibodies against human mature EMAPII.

FIG. 7 depicts an ELISA screen of eight antibodies against mouse proEMAPII.

FIG. 8 depicts an ELISA screen of eight antibodies against mouse mature EMAPII.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only and is not intended to be limiting.

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, analytical chemistry, immunology, and nucleic acid chemistry and hybridization, monoclonal antibody synthesis and biological and biophysical characterization are those well-known and commonly employed in the art. Standard techniques or modifications thereof are used for chemical syntheses and chemical analyses.

The articles “a” and “an” are used herein to refer to one or to more than one (i.e. to at least one) of the grammatical object of the article. By way of example, “an element” means one element or more than one element.

As used herein, the term “Endothelial monocyte activating polypeptide II” or “EMAPII” refers to the proinflammatory cytokine. EMAPII is released from cells as either a 34 kD pro-form (“pro-EMAPII”) or a 18 kDa mature protein upon proteolytic cleavage by proteases (“mature-EMAPII”). The human homolog of mature EMAPII comprises the sequence

SEQ ID NO: 12: SKPIDVSRLDLRIGCIITARKHPDADSLYVEEVDV
GEIAPRTVVSGLVNHVPLEQMQNRMVILLCNLKPAKMRGVLSQAMVMCAS
SPEKIEILAPPNGSVPGDRITFDAFPGEPDKELNPKKKIWEQIQPDLHT
NDECVATYKGVPFEVKGKGVCRAQTMSNSGIK.

The term “EMAPII” may be used to refer to both the pro and mature forms of this protein unless otherwise specified.

As used herein, the term “about” will be understood by persons of ordinary skill in the art and will vary to some extent on the context in which it is used. As used herein when referring to a measurable value such as an amount, a concentration, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “affinity” for a molecule towards another refers to the degree (or tightness) of binding between the two molecules. A higher affinity means tighter binding between the two molecules. Affinity can be quantified in terms of dissociation constant (or K_{d}), where a K_{d} value that is lower in magnitude (closer to zero) indicates a higher affinity.

An “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides, and particularly at the carboxy- or amino-terminus, can be modified by methylation, amidation, acetylation or substitution with other chemical groups which can change a peptide’s circulating half-life without adversely affecting activity of the peptide. Additionally, a disulfide linkage may be present or absent in the peptides.

The term “antibody,” as used herein, refers to an immunoglobulin molecule able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks the biological activity of the antigen.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. α and β light chains refer to the two major antibody light chain isotypes.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated or synthesized or can be derived from a biological sample. Such a biological sample can include, but is not limited to a tissue sample, a tumor sample, a cell or a biological fluid.

A “coding region” of a gene consists of the nucleotide residues of the coding strand of the gene and the nucleotides of the non-coding strand of the gene that are homologous with or complementary to, respectively, the coding region of an mRNA molecule produced by transcription of the gene.

A “coding region” of an mRNA molecule also consists of the nucleotide residues of the mRNA molecule that are matched with an anti-codon region of a transfer RNA molecule during translation of the mRNA molecule or that encode a stop codon. The coding region may thus include nucleotide residues corresponding to amino acid residues not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

As used herein, the terms “conservative variation” or “conservative substitution” as used herein refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine, and the like.

The term “delivery vehicle” is used herein as a generic reference to any delivery vehicle capable of delivering a compound to a subject, including, but not limited to, parenteral delivery vehicles.

The term “DNA” as used herein is defined as deoxyribonucleic acid.

“Effective amount” or “therapeutically effective amount” are used interchangeably herein, and refer to an amount of a compound, formulation, material, or composition, as described herein, effective to achieve a particular biological result. Such results may include, but are not limited to, treatment of a disease or condition as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “EMAPII mediated disease” means any disease in a subject in which excessive levels of EMAPII plays a role where treatment is affected by reducing the level of EMAPII in the subject.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

An “individual”, “patient” or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “protein”, “peptide” and “polypeptide” are used interchangeably and refer to a compound comprised of amino acid residues covalently linked by peptide bonds. The term “peptide bond” means a covalent amide linkage formed by loss of a molecule of water between the carboxyl group of one amino acid and the amino group of a second amino acid. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Proteins” include, for example, biologically active fragments, substantially homologous proteins, oligopeptides, homodimers, heterodimers, variants of proteins, modified proteins, derivatives, analogs, and fusion proteins, among others. The proteins include natural proteins, recombinant proteins, synthetic proteins, or a combination thereof. A protein may be a receptor or a non-receptor.

The term “recombinant DNA” as used herein is defined as DNA produced by joining pieces of DNA from different sources.

The term “recombinant polypeptide” as used herein is defined as a polypeptide produced by using recombinant DNA methods.

The term “RNA” as used herein is defined as ribonucleic acid.

The term “therapeutic” as used herein means a treatment and/or prophylaxis.

The term to “treat,” as used herein, means reducing the frequency with which symptoms are experienced by a subject or administering an agent or compound to reduce the frequency and/or severity with which symptoms are experienced. As used herein, “alleviate” is used interchangeably with the term “treat.”

As used herein, “treating a disease, disorder or condition” means reducing the frequency or severity with which a symptom of the disease, disorder or condition is experienced by a subject. Treating a disease, disorder or condition may or may not include complete eradication or elimination of the symptom.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, adenoviruses, and adeno-associated viruses) that incorporate the recombinant polynucleotide.

A “vector” is a composition of matter that comprises a gene and that may be used to deliver the gene to the interior of a cell. Vector refers to any plasmid containing the gene that is capable of moving foreign sequences into the genomes of a target organism or cell.

As used herein, the term “fragment” as applied to a nucleic acid, is less than the whole.

The following abbreviations are used herein: V_{H,} heavy chain variable region; V_{L,} light chain variable region; C_{H,} heavy chain constant region; C_{L,} light chain constant region.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention provides a humanized monoclonal antibody that recognizes pro- and mature EMAPII. The antibody comprises one of four light chain variable regions and one of five heavy chain variable regions. The invention further provides a chimeric anti-EMAPII antibody. Pharmaceutical compositions for delivery of the antibodies are further disclosed as are methods of treating diseases by targeting EMAPII with the antibody of the invention.

### Antibodies, Vectors and Compositions Comprising Antibodies

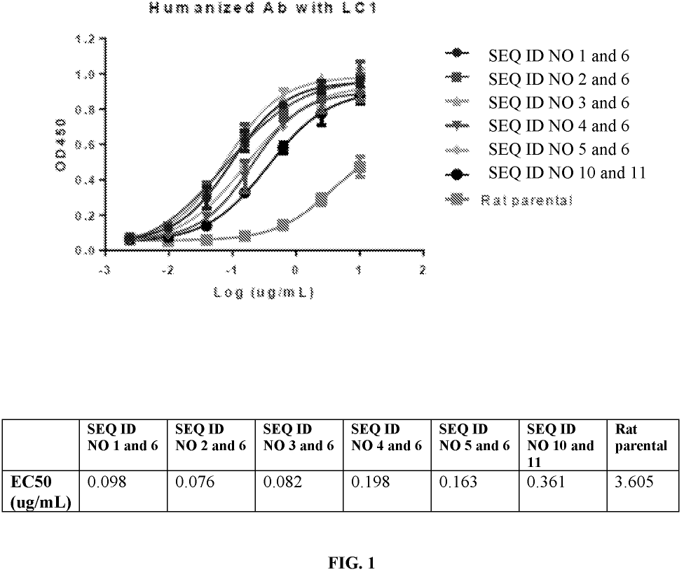

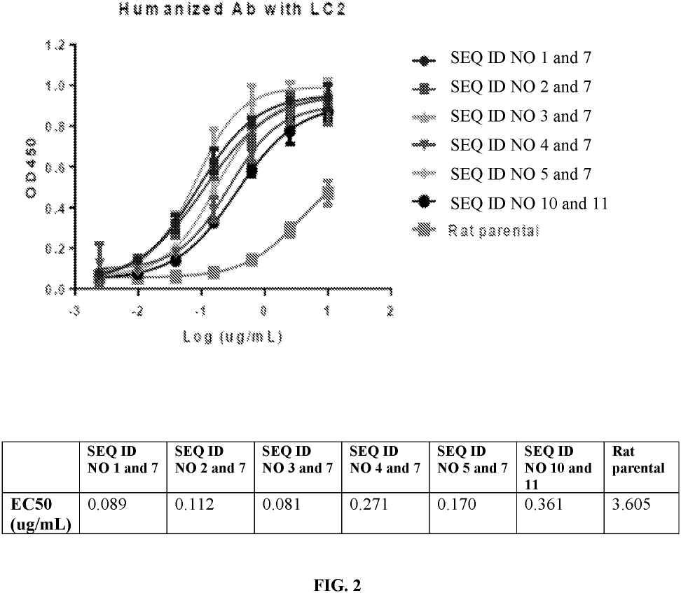

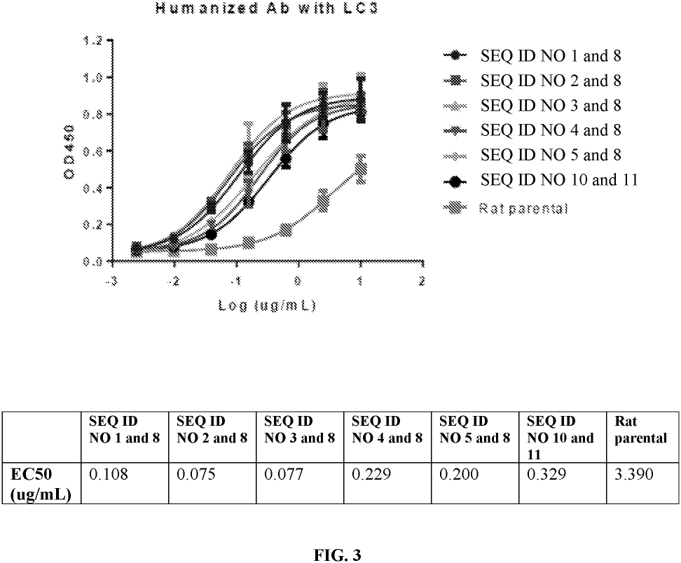

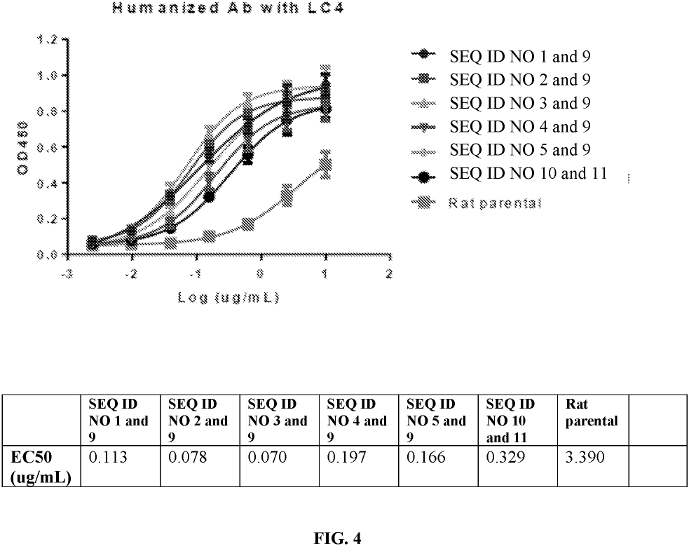

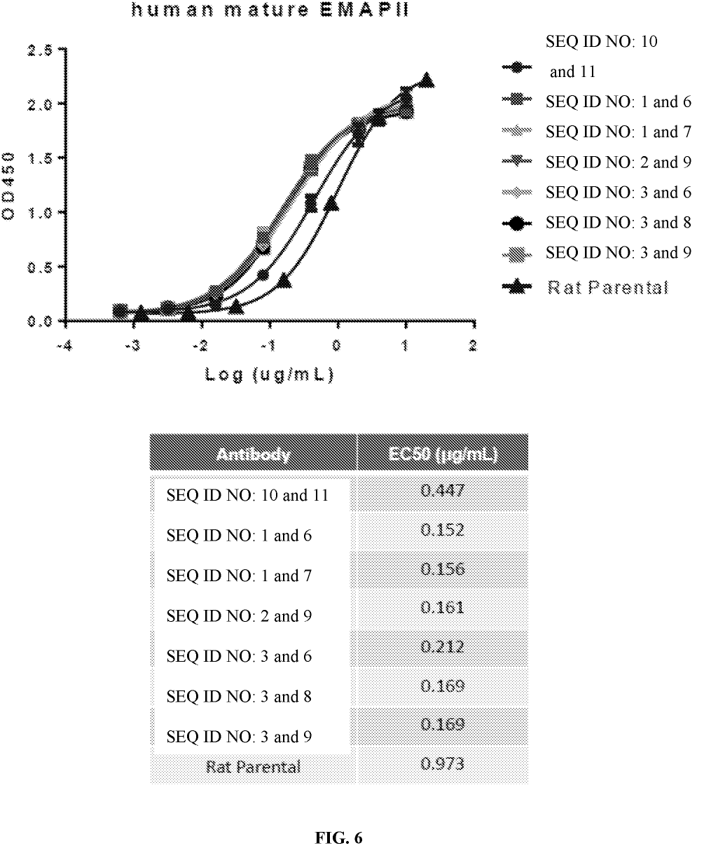

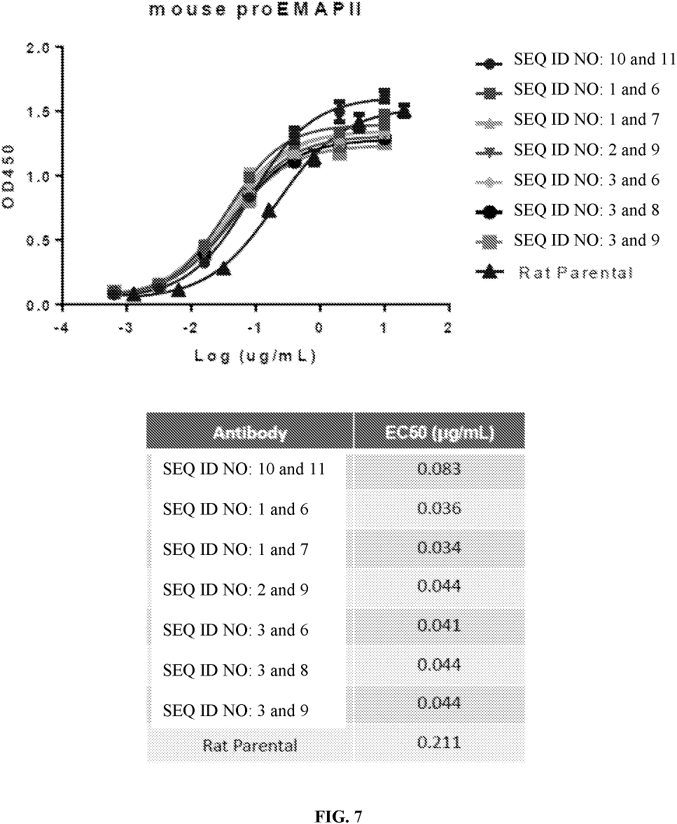

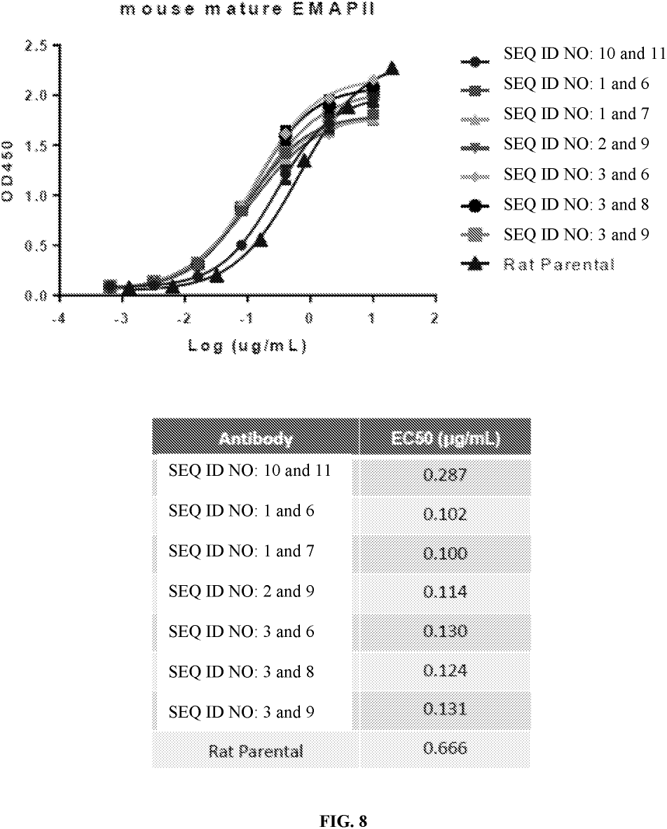

In one aspect, the invention provides a humanized anti-EMAPII antibody. The antibody comprises a heavy chain variable domain comprising an amino acid sequence having 90% or greater sequence identity to any one of SEQ ID NO: 1 to SEQ ID NO: 5 and a light chain variable domain comprising an amino acid sequence having 90% or greater sequence identity to any one of SEQ ID NO: 6 to SEQ ID NO: 9. As shown in FIGS. 1-8 and Example 1, the antibodies disclosed herein bind to pro- and mature EMAPII.

H4424 (Humanized HC1) SEQ ID NO: 1 MDPKGSLSWRILLFL
SLAFELSYGEVQLVESGGGLVQPGGSLKLSCAASGFTFSDAAMYWVRQAS
GKGLEWVGRIRTKPNNYATYYAASVKGRFTISRDDSKNTAYLQMNSLKTE
DTAVYYCTSWSYDFDYWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPG

H4425 (Humanized HC2) SEQ ID NO: 2 MDPKGSLSWRILLFL
SLAFELSYGAVQLVESGGGLVQPGGSLKLSCAASGFTFSDAAMYWVRQAS
GKGLEWVARIRTKPNNYATYYAASVKGRFTISRDDSKNTVYLQMNSLKTE
DTAVYYCTSWSYDFDYWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPG

H4426 (Humanized HC3) SEQ ID NO: 3 MDPKGSLSWRILLFL
SLAFELSYGAVHLVESGGGLVQPGGSLKLSCAASGFTFSDAAMYWVRQAS
GKGLEWVARIRTKPNNYATYYAASVKGRFTISRDDSKNTVYLQMNSLKTE
DTAVYYCTSWSYDFDYWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPG

H4427 (Humanized HC4) SEQ ID NO: 4 MDPKGSLSWRILLFL
SLAFELSYGEVQLVESGGGLVQPGGSLKLSCAASGFTFSDAAIYWVRQAS
GKGLEWVARIRTKPNNYATYYAASVKGRFTISRDDSNSTAYLQMNSLKTE
DTAVYYCTSWSYDFDYWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPG

H4428 (Humanized HC5) SEQ ID NO: 5 MDPKGSLSWRILLFL
SLAFELSYGAVQLVESGGGLVQPGGSLKLSCAASGFTFSDAAMYWVRQAS
GKGLEWVARIRTKPNNYATYYAASVKGRFTISRDDSNSTAYLQMNSLKTE
DTAVYYCTSWSYDFDYWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPG

L4424 (Humanized LC1) SEQ ID NO: 6 METDTLLLWVLLLWV
PGSTGDIVMTQSPLSLPVTPGEPASISCRSSKSLLHSSGKTYLNWYLQKP
GQSPQLLIYWMSNRASGVPDRFSGSGSGTDFTLKISRVEAEDVGVYYCQQ
FLEYPLTFGQGTKLEIKRTVAAPSVFIFPPSDEQLKSGTASVVCLLNNFY
PREAKVQWKVDNALQSGNSQESVTEQDSKDSTYSLSSTLTLSKADYEKHK
VYACEVTHQGLSSPVTKSFNRGEC

L4425 (Humanized LC2) SEQ ID NO: 7 METDTLLLWVLLLWV
PGSTGDIVMTQSPLSLPVTPGEPASISCRSSKSLLHSSGKTYLNWYLQKP
GQSPQLLIYWMSTRASGVPDRFSGSGSGTDFTLKISRVEAEDVGVYYCQQ
FLEYPLTFGQGTKLEIKRTVAAPSVFIFPPSDEQLKSGTASVVCLLNNFY
PREAKVQWKVDNALQSGNSQESVTEQDSKDSTYSLSSTLTLSKADYEKHK
VYACEVTHQGLSSPVTKSFNRGEC

L4426 (Humanized LC3) SEQ ID NO: 8 METDTLLLWVLLLWV
PGSTGDIVMTQSPLSLPVTPGEPASISCRSSKSLLHSSGKTYLNWYLQKP
GQSPQLLIYWMSTRASGVPDRFSGSGSGTDFTLKISRVEAEDVGVYYCQQ
FLEYPLTFGGGTKVEIKRTVAAPSVFIFPPSDEQLKSGTASVVCLLNNFY
PREAKVQWKVDNALQSGNSQESVTEQDSKDSTYSLSSTLTLSKADYEKHK
VYACEVTHQGLSSPVTKSFNRGEC

L4427 (Humanized LC4) SEQ ID NO: 9 METDTLLLWVLLLWV
PGSTGDIQMTQSPLSLPVTPGEPASISCRSSKSLLHSSGKTYLNWYLQKP
GQSPQLLIYWMSTRASGVPDRFSGSGSGTDFTLKISRVEAEDVGVYYCQQ
FLEYPLTFGGGTKVEIKRTVAAPSVFIFPPSDEQLKSGTASVVCLLNNFY
PREAKVQWKVDNALQSGNSQESVTEQDSKDSTYSLSSTLTLSKADYEKHK
VYACEVTHQGLSSPVTKSFNRGEC

In various embodiments, one or both of the heavy and light chain variable region of the humanized anti-EMAPII antibody comprises an amino acid sequence having 95% or greater, 96% or greater, 97% or greater, 98% or greater, 99% or greater or 100% sequence identity to SEQ ID NO: 1 to SEQ ID NO: 5, and to SEQ ID NO: 6 to SEQ ID NO: 9, respectively.

In another aspect, the invention provides a chimeric anti-EMAPII antibody, comprising a heavy chain variable domain with 90% or greater sequence identity to SEQ ID NO: 10 and a light chain variable domain with 90% or greater sequence identity to SEQ ID NO: 11.

MHC1309HC.4 SEQ ID NO: 10 AVHLVESGGGFVQPTESLKISCAA
SGFTFSDAAMYWVRQAPGKGLEWVARIRTKPNNYATYYADSVKGRFTISR
DDSKSMVYLQMDNLKTEDTAMYYCTSWSYDFDYWGQGVMVTVSS

MHC1309LC.1 SEQ ID NO: 11 DIVMTQGALPNPVPSGESASITCQ
SSKSLLHSSGKTYLNWYLQRPGQSPHLLIYWMSTRASGVSDRLSGSGSGT
DFTLKISSVEAEDVGVYYCQQFLEYPLTFGSGTKLEIK

In various embodiments, one or both of the heavy and light chain variable regions of the chimeric anti-EMAPII antibody comprises an amino acid sequence with 95% or greater, 96% or greater, 97% or greater, 98% or greater, 99% or greater or 100% sequence identity to SEQ ID NO: 10 and SEQ ID NO: 11, respectively.

In another aspect, the invention provides a recombinant vector, comprising a nucleic acid encoding an amino acid sequence set forth in any one of SEQ ID NO: 1 to SEQ ID NO: 11. In various embodiments, the vector comprises a nucleic acid encoding a humanized anti-EMAPII antibody heavy chain as set forth in any one of SEQ ID NO: 1 to SEQ ID NO:5. In various embodiments, the vector comprises a nucleic acid encoding a humanized anti-EMAPII antibody light chain as set forth in any one of SEQ ID NO: 6 to SEQ ID NO: 11. In various embodiments, the vector comprises a nucleic acid encoding a humanized anti-EMAPII antibody heavy chain as set forth in any one of SEQ ID NO: 1 to SEQ ID NO:5 and a humanized anti-EMAPII antibody light chain as set forth in any one of SEQ ID NO: 6 to SEQ ID NO:9. In various embodiments, In various embodiments, the nucleic acid is operably linked to a promoter. In various embodiments, the invention provides a cell transformed with the recombinant vector. The invention further provides a method of producing a humanized anti-EMAPII antibody heavy chain as set forth in any one of SEQ ID NO: 1 to SEQ ID NO: 5 and a humanized anti-EMAPII antibody light chain as set forth in any one of SEQ ID NO: 6 to SEQ ID NO: 9, comprising culturing the cell transformed with the vector under conditions in which the antibody is expressed.

In brief summary, the expression of natural or synthetic nucleic acids encoding antibodies is typically achieved by operably linking a nucleic acid encoding the antibody polypeptide or portions thereof to a promoter and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., Molecular Cloning: A Laboratory Manual, volumes 1-3 (3^{rd} ed., Cold Spring Harbor Press, NY 2001), and in other virology and molecular biology manuals. Viruses, which are useful as vectors include, but are not limited to, retroviruses, adenoviruses, adeno- associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the EF1alpha promoter. An additional example includes the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In order to assess the expression of an antibody polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co- transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter- driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., Molecular Cloning: A Laboratory Manual, volumes 1-3 (3^{rd} ed., Cold Spring Harbor Press, NY 2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about -20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In various embodiments, the invention provides a pharmaceutical composition comprising the antibody and at least one pharmaceutically acceptable excipient. The pharmaceutical composition may be in any form appropriate for the delivery of an antibody to a patient and the specifics of the pharmaceutical composition will vary based on the indication for which it is provided. Examples of suitable pharmaceutical excipients are disclosed elsewhere herein.

### Method of Treating Disease

EMAPII is associated with cigarette smoke induced lung injury as well as lung injury generally. As shown in FIGS. 1-8, the antibodies of the invention bind both pro- and mature-EMAPII. Accordingly, in various embodiments, the invention provides a method of treating chronic obstructive pulmonary disease (COPD), in particular emphysema, in a subject in need thereof, the method comprising administering to the subject a humanized anti-EMAPII antibody as disclosed herein in a pharmaceutical composition.

In various embodiments, the invention provides a method of treating emphysema in a subject in need thereof, the method comprising administering to the subject a humanized anti-EMAPII antibody as disclosed herein in a pharmaceutical composition.

In various embodiments, the invention provides a method of treating acute lung injury in a subject in need thereof, the method comprising administering to the subject a humanized anti-EMAPII antibody as disclosed herein in a pharmaceutical composition.

In various embodiments, the invention provides a method of treating bronchopulmonary dysplasia in a subject in need thereof, the method comprising administering to the subject a humanized anti-EMAPII antibody as disclosed herein in a pharmaceutical composition.

The anti-EMAPII antibody disclosed herein binds to and reduces the level of EMAPII available to interact with target cells. Accordingly, in various embodiments, the invention provides a method of treating an EMAPII mediated disease in a subject in need thereof, the method comprising administering to the subject a humanized anti-EMAPII antibody as disclosed herein in a pharmaceutical composition

### Administration/Dosage/Formulations

Administration of the compounds and/or compositions of the present invention to a patient, preferably a mammal, more preferably a human, may be carried out using known procedures, at dosages and for periods of time effective to treat the intended disease. An effective amount of the therapeutic compound necessary to treat the intended disease may vary according to factors such as the state of a disease or disorder in the patient; the age, sex, and weight of the patient; and the equipment used to detect the compound of the invention. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve treatment for a particular patient, composition, and mode of administration, without being toxic to the patient.

In certain embodiments, the compositions of the invention are formulated using one or more pharmaceutically acceptable excipients or carriers. In certain embodiments, the pharmaceutical compositions of the invention comprise an effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Compounds of the invention for administration may be in the range of from about 1 µg to about 10,000 mg, about 20 µg to about 9,500 mg, about 40 µg to about 9,000 mg, about 75 µg to about 8,500 mg, about 150 µg to about 7,500 mg, about 200 µg to about 7,000 mg, about 3050 µg to about 6,000 mg, about 500 µg to about 5,000 mg, about 750 µg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In certain embodiments, the dose of a compound of the invention is from about 1 mg and about 2,500 mg. In certain embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in certain embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans) buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer’s solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Additional Administration Forms

Additional dosage forms of this invention include dosage forms as described in U.S. Pats. Nos. 6,340,475; 6,488,962; 6,451,808; 5,972,389; 5,582,837; and 5,007,790. Additional dosage forms of this invention also include dosage forms as described in U.S. Pat. Applications Nos. 2003/0147952; 2003/0104062; 2003/0104053; 2003/0044466; 2003/0039688; and 2002/0051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, practice the claimed methods of the present invention. The following working examples therefore, specifically point out the preferred embodiments of the present invention and are not to be construed as limiting in any way the remainder of the disclosure.

### Example 1

### Cloning and Sequencing of Rat Hybridoma IgG Variable and Constant Regions

Rapid amplification of cDNA ends (RACE) was performed to amplify DNA for V_{H} and V_{L}, C_{H} and C_{L} regions. Positive clones were identified by gel electrophoresis. Positive DNA was cloned and sequenced. The DNA and amino acid sequences for IgG variable and constant regions was analyzed.

Humanization design of the parental antibody was performed using in silico analyses. The humanization process begins by generating a homology modeled antibody 3D structure and creating a profile of the parental antibody based on structure modeling. Acceptor frameworks to utilize were identified based on the overall sequence identity across the framework, matching interface position, similarly classed CDR canonical positions, and presence of N-glycosylation sites that would have to be removed. Two light chain (LC) and two heavy chain (HC) frameworks were selected for the humanization design.

Humanized antibodies were designed by creating multiple hybrid sequences that fuse select parts of the parental antibody sequence with the human framework sequences. Using the 3D model, these humanized sequences were methodically analyzed by eye and computer modeling to isolate the sequences that would most likely retain antigen binding. The goal was to maximize the amount of human sequence in the final humanized antibodies while retaining the original antibody specificity.

Three humanized light chains and five humanized heavy chains were designed based on two different heavy and light chain human acceptor frameworks (see Table 1). HC1 and LC1 utilize the first respective framework and contain the most human sequence with minimal parental antibody framework sequence. HC2, HC3 and LC2 use the same framework as before but contain additional parental sequences. HC4, HC5, LC3 and LC4 utilize the second respective framework and also contain additional parental sequences fused with the human framework.

TABLE 1

Humanized chain information table

| | | |
|---|---|---|
| Chain Name | Chain Type | Acceptor Framework |
| H4423 (chimeric parental) SEQ ID NO: 10 | Heavy chain | |
| L4423 (chimeric parental) SEQ ID NO: 11 | Light chain | |
| H4424 (Humanized HC 1) SEQ ID NO: 1 | Heavy chain | HC framework 1 |
| H4425 (Humanized HC 2) SEQ ID NO: 2 | Heavy chain | HC framework 1 |
| H4426 (Humanized HC 3) SEQ ID NO: 3 | Heavy chain | HC framework 1 |
| H4427 (Humanized HC 4) SEQ ID NO: 4 | Heavy chain | HC framework 2 |
| H4428 (Humanized HC 5) SEQ ID NO: 5 | Heavy chain | HC framework 2 |
| L4424 (Humanized LC 1) SEQ ID NO: 6 | Light chain | LC framework 1 |
| L4425 (Humanized LC 2) SEQ ID NO: 7 | Light chain | LC framework 1 |
| L4426 (Humanized LC 3) SEQ ID NO: 8 | Light chain | LC framework 2 |
| L4427 (Humanized LC 4) SEQ ID NO: 9 | Light chain | LC framework 2 |

### Calculation of Humanness Scores of Humanized Chains

The humanness scores for the monoclonal antibodies were calculated according to Gao, S.H., Huang, K., Tu, H., and Adler, A.S. 2013, Monoclonal antibody humanness score and its applications. BMC Biotechnology, 13:55. This score represents how human-like an antibody variable region sequence is in terms of reduced antigenicity in humans, which is an important factor when humanizing antibodies. The humanness scores for the parental and humanized antibodies are shown below. Based on this method, for heavy chains a score of 79 or above is indicative of a human-like antibody in terms of its antigenicity; for kappa light chains a score of 86 or above is indicative of a human-like antibody in terms of its antigenicity.

TABLE 2

Humanness scores of heavy chains

| VH | Full-length (Framework+CDR) Cutoff = 79 | Framework Only Cutoff = 84 |
|---|---|---|
| Parental H4423 SEQ ID NO: 10 | 74.0 | 74.0 |
| H4424 SEQ ID NO: 1 | 87.9 | 91.5 |
| H4425 SEQ ID NO: 2 | 85.4 | 87.9 |
| H4426 SEQ ID NO: 3 | 84.5 | 86.6 |
| H4427 SEQ ID NO: 4 | 85.6 | 89.3 |
| H4428 SEQ ID NO: 5 | 85.5 | 88.3 |

TABLE 3

Humanness scores of light chains

| VL | Full-length (Framework+CDR) Cutoff = 86 | Framework Only Cutoff = 90 |
|---|---|---|
| Parental L4423 SEQ ID NO: 11 | 76.0 | 81.9 |
| L4424 SEQ ID NO: 6 | 88.3 | 98.7 |
| L4425 SEQ ID NO: 7 | 87.6 | 98.7 |
| L4426 SEQ ID NO: 8 | 88.2 | 99.3 |
| L4427 SEQ ID NO: 9 | 87.4 | 98.3 |

### Construction of Humanized Antibodies

Full-length antibody genes were constructed by first synthesizing the variable region sequences. The sequences were optimized for expression in mammalian cells. These variable region sequences were then cloned into expression vectors that already contain human Fc domains. In addition, for comparison the variable regions of the parental heavy and light chains were constructed as full-length chimeric chains using the same backbone Fc sequences.

### Small Scale Production

Humanized antibodies underwent 0.03 liter production. The chimeric parental antibody was also scaled-up for direct comparison. Plasmids for the indicated heavy and light chains were transfected into HEK293 cell in suspension using chemically defined media in the absence of serum to make the antibodies. Specifically, antibody sequences were gene synthesized and cloned into proprietary expression vector pLEV123. Whole antibodies in the conditioned media were purified using MabSelect SuRe Protein A medium (GE Healthcare).

TABLE 3

Antibodies produced in HEK293 cells transiently

| Antibody: | Heavy Chain | Light Chain | Titer (mg/L) |
|---|---|---|---|
| Chimeric Parental | SEQ ID NO: 10 | SEQ ID NO: 11 | 20 |
| Humanized HC1 + LC1 | SEQ ID NO: 1 | SEQ ID NO: 6 | 41 |
| Humanized HC1 + LC2 | SEQ ID NO: 1 | SEQ ID NO: 7 | 42 |
| Humanized HC1 + LC3 | SEQ ID NO: 1 | SEQ ID NO: 8 | 37 |
| Humanized HC1 + LC4 | SEQ ID NO: 1 | SEQ ID NO: 9 | 36 |
| Humanized HC2 + LC1 | SEQ ID NO: 2 | SEQ ID NO: 6 | 35 |
| Humanized HC2 + LC2 | SEQ ID NO: 2 | SEQ ID NO: 7 | 39 |
| Humanized HC2 + LC3 | SEQ ID NO: 2 | SEQ ID NO: 8 | 38 |
| Humanized HC2 + LC4 | SEQ ID NO: 2 | SEQ ID NO: 9 | 41 |
| Humanized HC3 + LC1 | SEQ ID NO: 3 | SEQ ID NO: 6 | 52 |
| Humanized HC3 + LC2 | SEQ ID NO: 3 | SEQ ID NO: 7 | 39 |
| Humanized HC3 + LC3 | SEQ ID NO: 3 | SEQ ID NO: 8 | 45 |
| Humanized HC3 + LC4 | SEQ ID NO: 3 | SEQ ID NO: 9 | 46 |
| Humanized HC4 + LC1 | SEQ ID NO: 4 | SEQ ID NO: 6 | 53 |
| Humanized HC4 + LC2 | SEQ ID NO: 4 | SEQ ID NO: 7 | 51 |
| Humanized HC4 + LC3 | SEQ ID NO: 4 | SEQ ID NO: 8 | 41 |
| Humanized HC4 + LC4 | SEQ ID NO: 4 | SEQ ID NO: 9 | 45 |
| Humanized HC5 + LC1 | SEQ ID NO: 5 | SEQ ID NO: 6 | 56 |
| Humanized HC5 + LC2 | SEQ ID NO: 5 | SEQ ID NO: 7 | 46 |
| Humanized HC5 + LC3 | SEQ ID NO: 5 | SEQ ID NO: 8 | 46 |
| Humanized HC5 + LC4 | SEQ ID NO: 5 | SEQ ID NO: 9 | 56 |

### Binding ELISA of Humanized Antibodies

Dose dependent binding ELISA of 20 humanized antibodies, the chimeric parental antibody and rat parental antibody was performed. ELISA plate was coated with human pro-EMAPII in phosphate buffered saline (PBS) at 2 µg/mL at 4° C. overnight. All antibodies were diluted by a 7 point, 4 fold serial dilution starting at 10 µg/mL with 0.05% phosphate buffered saline tween (PBST), the last point being PBST alone. Antibodies were incubated on pre-blocked ELISA plate for 1.5 hours followed by 3 times wash with 0.05% PBST. Secondary antibody (horseradish peroxidase (HRP)-anti-human IgG Fc or HRP-anti-rat IgG Fc) was diluted 5000x with HRP assay diluent and incubated for 1 hour at room temperature. The plate was then washed 4 times and TMB substrate was added. Color was developed at room temperature for 5 minutes before the reaction was stopped by 1N HCl. Binding curves are shown in FIGS. 1-4.

### Binding to Pro- and Mature EMAPII

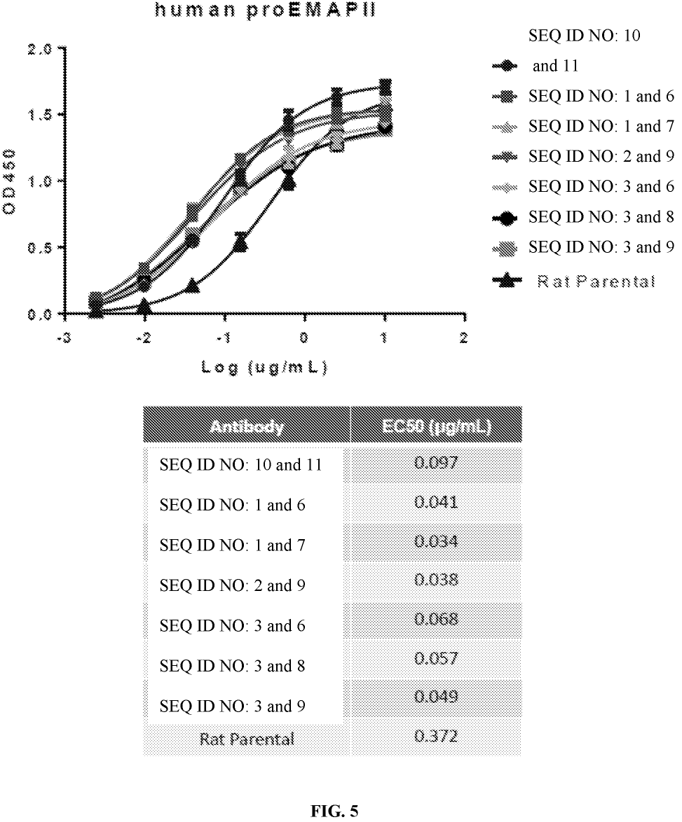

Dose dependent binding ELISA of 20 antibodies were performed and the results are shown in FIGS. 5-8. The coating of antigen was 2 µg/mL at 4° C. overnight. Blocking with 2% BSA in PBS, room temperature 1.5 hours. Primary incubation was performed at room temperature for 1 hour at 10 µg/mL, 1:4 dilution. The detection antibody was Goat-anti-Human for humanized and chimeric antibodies; Goat-anti-Rat for Rat Parental. RT 1 hours (1:10000 dilution).

### List of Enumerated Embodiments

Embodiment 1. A humanized anti-endothelial monocyte activating polypeptide II (anti-EMAPII) antibody, comprising:
a heavy chain variable domain having an amino acid sequence with 90% or greater sequence identity to any one of SEQ ID NO: 1 to SEQ ID NO: 5; and
a light chain variable domain having an amino acid sequence with 90% or greater sequence identity to any one of SEQ ID NO: 6 to SEQ ID NO: 9.

Embodiment 2. The antibody according to embodiment 1, wherein the heavy chain variable domain has an amino acid sequence with 95% or greater sequence identity to any one of SEQ ID NO: 1 to SEQ ID NO: 5; and

the light chain variable domain has an amino acid sequence with 95% or greater sequence identity to any one of SEQ ID NO: 6 to SEQ ID NO: 9.

Embodiment 3. The antibody according to embodiment 1, wherein the heavy chain variable domain has an amino acid sequence with 99% or greater sequence identity to any one of SEQ ID NO: 2 or SEQ ID NO: 3; and

the light chain variable domain has an amino acid sequence with 99% or greater sequence identity to any one of SEQ ID NO: 6 or SEQ ID NO: 9.

Embodiment 4. A humanized anti-endothelial monocyte activating polypeptide II (anti-EMAPII) antibody, comprising a heavy chain variable domain having an amino acid sequence with 90% or greater sequence identity to any one of SEQ ID NO: 1 to SEQ ID NO: 5.

Embodiment 5. The antibody according to Embodiment 4, wherein the heavy chain variable domain has an amino acid sequence with 95% or greater sequence identity to any one of SEQ ID NO: 2 or SEQ ID NO: 3.

Embodiment 6. A humanized anti-endothelial monocyte activating polypeptide II (anti-EMAPII) antibody, comprising a light chain variable domain having an amino acid sequence with 90% or greater sequence identity to any one of SEQ ID NO: 6 to SEQ ID NO: 9.

Embodiment 7. The antibody according to Embodiment 6, wherein the light chain variable domain has an amino acid sequence with 95% or greater sequence identity to any one of SEQ ID NO: 6 or SEQ ID NO: 9.

Embodiment 8. A chimeric anti-endothelial monocyte activating polypeptide II (anti-EMAPII) antibody, comprising a heavy chain variable domain with 90% or greater sequence identity to SEQ ID NO: 10 and a light chain variable domain with 90% or greater sequence identity to SEQ ID NO: 11.

Embodiment 9. The antibody according to Embodiment 8, wherein the heavy chain variable domain has 95% or greater sequence identity to SEQ ID NO: 10 and the light chain variable domain has 95% or greater sequence identity to SEQ ID NO: 11.

Embodiment 10. A recombinant vector, comprising a nucleic acid encoding an amino acid sequence set forth in any one of SEQ ID NO: 1 to SEQ ID NO: 11.

Embodiment 11. A cell transformed with the recombinant vector according to Embodiment 10. Embodiment 12. A method of producing an antibody, comprising culturing the transformed cell of Embodiment 11 under conditions in which the antibody is expressed.

Embodiment 13. A pharmaceutical composition comprising the antibody according to any one of Embodiments 1-9 and at least one pharmaceutically acceptable excipient.

Embodiment 14. A method of treating chronic obstructive pulmonary disease (COPD) in a subject in need thereof, the method comprising administering to the subject the pharmaceutical composition according to Embodiment 13.

Embodiment 15. A method of treating emphysema in a subject in need thereof, the method comprising administering to the subject the pharmaceutical composition according to Embodiment 13.

Embodiment 16. A method of treating bronchopulmonary dysplasia in a subject in need thereof, the method comprising administering to the subject the pharmaceutical composition according to Embodiment 13.

Embodiment 17. A method of treating endothelial monocyte activating polypeptide II (EMAPII) mediated disease in a subject in need thereof, the method comprising administering to the subject the pharmaceutical composition according to Embodiment 13.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A humanized anti-endothelial monocyte activating polypeptide II (anti-EMAPII) antibody, comprising:
a heavy chain variable domain having an amino acid sequence with 90% or greater sequence identity to any one of SEQ ID NO: 1 to SEQ ID NO: 5; and
a light chain variable domain having an amino acid sequence with 90% or greater sequence identity to any one of SEQ ID NO: 6 to SEQ ID NO: 9.

2. The antibody according to claim 1, wherein the heavy chain variable domain has an amino acid sequence with 95% or greater sequence identity to any one of SEQ ID NO: 1 to SEQ ID NO: 5; and
the light chain variable domain has an amino acid sequence with 95% or greater sequence identity to any one of SEQ ID NO: 6 to SEQ ID NO: 9.

3. The antibody according to claim 1, wherein the heavy chain variable domain has an amino acid sequence with 99% or greater sequence identity to any one of SEQ ID NO: 2 or SEQ ID NO: 3; and
the light chain variable domain has an amino acid sequence with 99% or greater sequence identity to any one of SEQ ID NO: 6 or SEQ ID NO: 9.

4. A humanized anti-endothelial monocyte activating polypeptide II (anti-EMAPII) antibody, comprising a heavy chain variable domain having an amino acid sequence with 90% or greater sequence identity to any one of SEQ ID NO: 1 to SEQ ID NO: 5.

5. The antibody according to claim 4, wherein the heavy chain variable domain has an amino acid sequence with 95% or greater sequence identity to any one of SEQ ID NO: 2 or SEQ ID NO: 3.

6. A humanized anti-endothelial monocyte activating polypeptide II (anti-EMAPII) antibody, comprising a light chain variable domain having an amino acid sequence with 90% or greater sequence identity to any one of SEQ ID NO: 6 to SEQ ID NO: 9.

7. The antibody according to claim 6, wherein the light chain variable domain has an amino acid sequence with 95% or greater sequence identity to any one of SEQ ID NO: 6 or SEQ ID NO: 9.

8. A chimeric anti-endothelial monocyte activating polypeptide II (anti-EMAPII) antibody, comprising a heavy chain variable domain with 90% or greater sequence identity to SEQ ID NO: 10 and a light chain variable domain with 90% or greater sequence identity to SEQ ID NO: 11.

9. The antibody according to claim 8, wherein the heavy chain variable domain has 95% or greater sequence identity to SEQ ID NO: 10 and the light chain variable domain has 95% or greater sequence identity to SEQ ID NO: 11.

10. A recombinant vector, comprising a nucleic acid encoding an amino acid sequence set forth in any one of SEQ ID NO: 1 to SEQ ID NO: 11.

11. A cell transformed with the recombinant vector according to claim 10.

12. A method of producing an antibody, comprising culturing the transformed cell of claim 11 under conditions in which the antibody is expressed.

13. A pharmaceutical composition comprising the antibody according to claim 1 and at least one pharmaceutically acceptable excipient.

14. A method of treating chronic obstructive pulmonary disease (COPD) in a subject in need thereof, the method comprising administering to the subject the pharmaceutical composition according to claim 13.

15. A method of treating emphysema in a subject in need thereof, the method comprising administering to the subject the pharmaceutical composition according to claim 13.

16. A method of treating bronchopulmonary dysplasia in a subject in need thereof, the method comprising administering to the subject the pharmaceutical composition according to claim 13.

17. A method of treating endothelial monocyte activating polypeptide II (EMAPII) mediated disease in a subject in need thereof, the method comprising administering to the subject the pharmaceutical composition according to claim 13.
